(19) United States
(12) Patent Application Publication
(10) Pub. No.: US 2016/0025619 A1
(43) Pub. Date: Jan. 28, 2016

(54) METHOD FOR OPERATING A BEARING

(71) Applicant: Padelis Katsaros, Schweinfurt (DE)

(72) Inventor: Padelis Katsaros, Schweinfurt (DE)

(73) Assignee: AKTIEBOLAGET SKF, Goteborg (SE)

(21) Appl. No.: 14/807,044

(22) Filed: Jul. 23, 2015

(30) Foreign Application Priority Data
Jul. 24, 2014 (DE) 102014214486.8

(51) Int. Cl. G01N 21/25; F16C 33/66; G01N 33/28; C10M 159/00

(52) U.S. Cl. CPC G01N 21/25; C10M 159/00; F16C 33/6633; G01N 33/2888

(57) ABSTRACT

A method for operating a rolling-element bearing includes manufacturing a lubricating grease by mixing a first pigment having a first color with a first grease component to form a first colored grease component, mixing a second pigment having a second color different than the first color with a second grease component to form a second colored grease component, and mixing the first and second colored grease components to form a grease having a grease color. The method also includes lubricating the rolling element bearing with the grease, operating the bearing, measuring the color of the grease and providing a signal when the grease color becomes a predetermined grease color.

### CROSS-REFERENCE

This application claims priority to German patent application no. 10 2014 214 486.8 filed on Jul. 24, 2015, the contents of which are fully incorporated herein by reference.

### TECHNOLOGICAL FIELD

The invention is directed to a method for operating a bearing, in particular a rolling-element bearing, that includes introducing lubricating grease into the bearing.

### BACKGROUND

It is known to lubricate bearings, including rolling-element bearings, with grease. The state or condition of the lubricating grease is critical to keeping the bearing or the unit into which the bearing is installed functioning properly. That is, if the grease degrades sufficiently, it may cease to provide adequate lubrication, and the bearing and/or unit in which the bearing is installed may fail.

It is desirable to obtain information about the state or about the remaining service life of the lubricating grease. When such information is reliable and easily obtainable, it allows an optimal maintenance interval of a system to be determined. This helps avoid lubrication intervals that are too short because they increase cost and may waste grease. It also helps avoid lubrication intervals that are too long, since waiting too long to relubricate a bearing carries a high risk of bearing failure.

The current procedure for determining a lubricant state or condition has been to calculate or estimate the grease service life based on empirical knowledge. Alternatively, grease samples can be taken from the bearing unit and analyzed to determine the state of the lubricating grease. Both methods are relatively expensive and imprecise.

### SUMMARY

An aspect of the disclosure is therefore to provide a method by which it is possible to optimize relubrication intervals for lubricating grease in a bearing. The method allows a current lubricating-grease state to be determined in a simple and precise manner so that the bearing can be relubricated with lubricating grease at the optimal time.

The disclosed method includes the following steps:

a) mixing at least a first and a second grease component together to produce a lubricating grease, wherein the two grease components are each a different color or are mixed with color pigments of different colors;

b) operating the bearing including the lubricating grease, and detecting, continuously or at predetermined time intervals the color of the grease;

c) changing the lubricating grease or relubricating the bearing with new lubricating grease if the color test according to step b) shows that the color of the lubricating grease has turned a predetermined color.

The implementation of step b) here can be performed visually by a person. However, it is preferably performed using a color sensor. The monitoring process can thus be automated.

The color sensor is preferably placed inside the bearing.

In carrying out step a) above, a thickener and/or a soap is preferably used as the first grease component. A base oil is preferably used as the second grease component.

According to one embodiment of the disclosure, one grease component is or is colored yellow, and the other is or is colored blue.

The proposed method thus provides a simple method for determining a lubricating-grease state in a bearing based on a specific coloring of the grease base components.

Specifically, the grease service life is largely determined by the ability of the grease to release oil. After a certain period of operation the grease “bleeds out,” i.e., sufficient oil can no longer be provided for lubrication. To date, it has been difficult to determine when the grease has bled out.

Grease essentially comprises two components, namely, a thickener or soap as the first component and a base oil as the second component. The thickener binds the oil in the form of fine oil droplets and releases the oil when pressure and flexure or other shear forces act on the grease.

The disclosed method includes coloring the two components, the thickener and the base oil, using different color pigments. The resulting grease color is characteristic for the correct mixing ratio. Grease color changes during operation are preferably determined by a color sensor. Conclusions can be drawn about the lubricant state of the grease depending on whether the color of the grease shifts toward the original color of the thickener or toward the original color of the base oil.

For example, if the thickener is colored yellow and the base oil is colored blue, then the lubricating grease generated by mixing these components will be a certain shade of green. If during operation the thickener releases more and more oil, which also means a loss of blue color pigment, the initial shade of green will become more yellow. In the extreme case, namely if the grease is completely bled out, then the grease will be completely yellow, that is, essentially the color of the original, colored, thickener.

It can be determined, by calculation or empirically, what amount of color change indicates that a critical mix ration (i.e., too little base oil in the thickener) has been reached. When the predetermined color is detected, it is necessary to relubricate the bearing.

The method thus allows a lubricant change to be carried out when necessary, that is, when the grease is at the end of its useful life, rather than before or after the grease has reached the end of its useful life. This saves resources and protects the environment.

A monitoring of functioning is thus possible in particular with critical applications.

### BRIEF DESCRIPTION OF THE DRAWINGS

An exemplary embodiment of the invention is depicted in the drawings.

FIG. 1 schematically shows the mixing a first component and a second component to form a grease.

FIG. 2 is a schematic sectional side elevational view illustrating a method of monitoring grease in a bearing.

### DETAILED DESCRIPTION

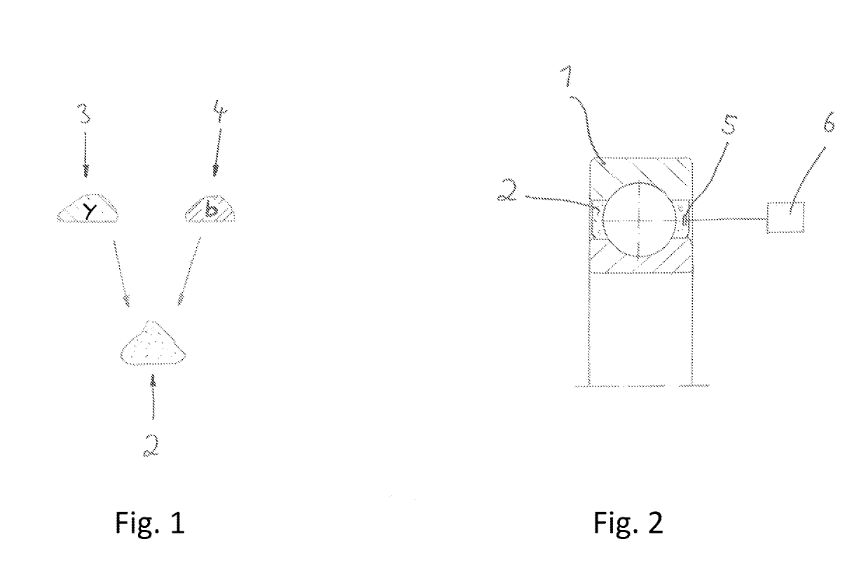

FIG. 1 schematically shows a grease 2 that is produced by mixing a first grease component 3 and a second grease component 4. For illustration purposes, the grease is made from only two components: a thickener or soap 3 and a base oil 4. However, a typical grease could include additives as well. The two components 3 and 4 each have a different color. The first component 3 is yellow or has yellow pigment added to it to make it yellow, and the second component 4 is blue or has blue pigment added to it to make it blue. The disclosure is not limited to these pigment colors, and different pigment colors could be used instead. Furthermore, the pigments are preferably selected such that the first pigment, the yellow pigment in the above example, is highly dissolvable in the thickener or soap 3, while the second component, the blue component in the above example, is highly dissolvable in the base oil 4. This may be accomplished, for example, by selecting a polar material for the yellow pigment and a non-polar material for the blue component. Alternately, the yellow pigment can be an ionic component while the blue pigment is non-ionic. Persons of ordinary skill in the art will appreciate the types of pigment that are most appropriate based on the properties of the particular thickener or soap 3 and base oil 4 that are used to form the grease 2.

The mixed components 3, 4 constitute the lubricating grease 2, which—as a mixture of blue and yellow components—is green.

This grease 2 is—as illustrated in FIG. 2—introduced into a rolling-element bearing 1 to provide lubrication for the rolling-element bearing 1.

The rolling-element bearing 1 includes a color sensor 5, which (via cable or wirelessly) is connected to an evaluating unit 6. The color sensor 5 measures the wavelength of light reflected by the lubricating grease, which wavelength determines the apparent color of the grease to an observer. The evaluating unit 6 may comprise, for example, a general purpose computer including a CPU and memory or any other device having a CPU or other computer chip configured to receive an input signal from the color sensor indicative of the wavelength of light measured and produce an output signal that indicates either the particular wavelength of light that has been detected or that a predetermined wavelength of light has been detected.

By measuring the wavelength of light reflected by the lubricating grease, the evaluating unit 6 can detect changes in the color of the lubricating grease and produce an output signal when a particular color or wavelength of reflected light is detected.

In the unused state, when the grease is still a shade of green, the wavelength range of the light reflected by the grease falls approximately in the range from 560 nm to 490 nm. If during operation the lubricating grease “bleeds out,” some amount of the blue base oil is lost and thus no longer contributes to the color of the lubricating grease 2. The grease 2 thus becomes more yellow (a yellow-green, for example). That is, the reflected light detected by the color sensor 5 now falls in a range of wavelengths from about 590 nm to 560 nm.

Accordingly the color sensor 5 or the evaluating unit 6 can deliver a message about the state of the lubricating grease when a color change is detected. For example, the evaluating unit may produce a visible or audible signal to indicate that the grease needs to be changed, i.e., that the wavelength detected by the color sensor has risen to above 560 nm. Alternately, the evaluating unit 6 may produce an output indicative of the actual color of the grease or the actual wavelength of the reflected light detected so that it can be determined how far the color of the grease has deviated from its initial color and/or how closely the color of the grease is approaching the predetermined color that indicates that the grease has worn out.

In summary, it can be said that the base oil of the lubricating grease and the grease soap are colored with different color pigments. The resulting color is characteristic of the correct mixing ratio between grease soap and base oil. A change in the color that occurs during operation of the bearing can be automatically detected by a color sensor. Conclusions regarding the state or condition of the lubricating grease can be drawn based on the direction in which the color changes, e.g., toward the color of the thickener or soap 3 or toward the color of the base oil 4.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above may be utilized separately or in conjunction with other features and teachings to provide improved methods of operating bearings.

Moreover, combinations of features and steps disclosed in the above detailed description may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

### REFERENCE NUMBER LIST

1 Bearing (rolling-element bearing)
2 Lubricating grease
3 First grease component (thickener / soap)
4 Second grease component (base oil)
5 Color sensor
6 Evaluating unit
y Yellow
b Blue

What is claimed is:

1. A method for operating a rolling-element bearing, comprising:
manufacturing a lubricating grease by mixing a first pigment having a first color with a first grease component to form a first colored grease component, mixing a second pigment having a second color different than the first color with a second grease component to form a second colored grease component, and mixing the first and second colored grease components to form a grease having a grease color;
lubricating the rolling element bearing with the grease;
operating the bearing;
measuring the color of the grease; and
providing a signal when the grease color becomes a predetermined grease color.

2. The method according to claim 1, wherein measuring the grease color comprises measuring a wavelength of light reflected from the grease.

3. The method according to claim 2, wherein measuring the grease color comprises measuring the grease color at predetermined time intervals.

4. The method according to claim 1 including changing the lubricating grease or relubricating the bearing after the signal that the grease has become the predetermined color is provided.

5. The method according to claim 1, wherein measuring the color of the grease comprises measuring the color of the grease using a color sensor.

6. The method according to claim 1, wherein measuring the color of the grease comprises measuring the color of the grease using a color sensor inside the bearing.

7. The method according to claim 1, wherein the first component comprises a thickener or a soap.

8. The method according to claim 7, wherein the second component comprises a base oil.

9. The method according to claim 8, wherein the first color is yellow and the second color is blue.

10. The method according to claim 1, wherein the first color is yellow and the second color is blue.

11. The method according to claim 1,
wherein the first component comprises a thickener or a soap and the second component comprises a base oil,
wherein measuring a color of the grease comprises measuring a wavelength of light reflected from the grease using a color sensor located in the rolling-element bearing, and
relubricating the rolling-element bearing after the signal is provided.

12. A method for operating a rolling-element bearing, comprising:
lubricating the rolling element bearing with a grease comprising a first grease component having a first color and a second grease component having a second color different than the first color;
operating the bearing;
measuring a color of the grease; and
providing a signal when the grease color becomes a predetermined grease color.

13. The method according to claim 12, including mixing a first pigment with the first grease component to produce the first grease component having the first color.

14. The method according to claim 13, including mixing a second pigment with the second grease component to produce the second grease component having the second color.

15. The method according to claim 14, wherein the first color is yellow and the second color is blue.

16. The method according to claim 12, wherein the first color is yellow and the second color is blue.
